(19) United States
(12) Patent Application Publication
(10) Pub. No.: US 2024/0189621 A1
(43) Pub. Date: Jun. 13, 2024

(54) METHODS AND SYSTEMS FOR RADIOTHERAPY PLAN VERIFICATION

(71) Applicant: SHANGHAI UNITED IMAGING HEALTHCARE CO., LTD., Shanghai (CN)

(72) Inventors: Wei HAN, Shanghai (CN); Yanfang LIU, Shanghai (CN); Zhao JIN, Shanghai (CN)

(73) Assignee: SHANGHAI UNITED IMAGING HEALTHCARE CO., LTD., Shanghai (CN)

(21) Appl. No.: 18/534,593

(22) Filed: Dec. 9, 2023

(30) Foreign Application Priority Data
Dec. 9, 2022 (CN) 202211582577.9

(51) Int. Cl. A61N 5/10; G16H 20/40; G16H 50/30

(52) U.S. Cl. CPC A61N 5/103; A61N 5/1049; G16H 20/40; G16H 50/30

(57) ABSTRACT

Systems and methods for radiotherapy plan verification are provided. The systems may obtain target radiotherapy plans which need to be verified. Further, the systems may generate a radiotherapy plan temporary list based on the target radiotherapy plans and verify the target radiotherapy plans in batches based on the radiotherapy plan temporary list using at least one verification means of at least one radiotherapy device.

### CROSS REFERENCE TO RELATED APPLICATIONS

This application claims priority to Chinese Patent Application No. 202211582577.9 filed on Dec. 9, 2022, the entire contents of which are hereby incorporated by reference.

### TECHNICAL FIELD

The present disclosure relates to the medical field, and in particular, to methods and systems for radiotherapy plan verification.

### BACKGROUND

Radiotherapy is widely employed in clinical treatment for cancers and other conditions. With the continuous development of modern radiotherapy technology, intensity modulated radiation therapy (IMRT) technology is increasingly used in radiotherapy. Because the execution of IMRT is relatively complex, in order to ensure that the radiation the radiotherapy plan can accurately irradiate the patient's lesion, radiotherapy plan verification (Plan QA) needs to be carried out before the treatment delivery. Therefore, it is desirable to provide methods and systems for radiotherapy plan verification, to save the labor cost of radiotherapy and improve the radiotherapy efficiency.

### SUMMARY

An aspect of the present disclosure relates to a method for radiotherapy plan verification. The method is implemented on a computing device including at least one processor. The method includes obtaining target radiotherapy plans which need to be verified; generating a radiotherapy plan temporary list based on the target radiotherapy plans; and verifying the target radiotherapy plans in batches based on the radiotherapy plan temporary list using at least one verification means of at least one radiotherapy device.

In some embodiments, the obtaining target radiotherapy plans which need to be verified includes determining the target radiotherapy plans from a plurality of radiotherapy plans.

In some embodiments, the determining the target radiotherapy plans from the plurality of radiotherapy plans includes presenting, via a user terminal, the plurality of radiotherapy plans; and receiving, via the user terminal, a user instruction for selecting the target radiotherapy plans from the plurality of radiotherapy plans.

In some embodiments, the user terminal is further directed to present a filtering element for filtering the plurality of radiotherapy plans using one or more filtering conditions. The user instruction is input via the filtering element presented by the user terminal. The one or more filtering conditions include at least one of a treatment part, a treatment subject, or a treatment type.

In some embodiments, the determining the target radiotherapy plans from the plurality of radiotherapy plans includes obtaining status information of the at least one radiotherapy device; for each of the plurality of radiotherapy plans, determining planned radiotherapy parameters of the radiotherapy plan; and determining a risk coefficient of the radiotherapy plan based on the planned radiotherapy parameters and the status information of a corresponding radiotherapy device; and selecting the target radiotherapy plans from the plurality of radiotherapy plans based on the risk coefficients of the plurality of radiotherapy plans.

In some embodiments, the generating the radiotherapy plan temporary list based on the target radiotherapy plans includes for each of the target radiotherapy plans, determining a starting irradiation angle and an ending irradiation angle of radiation beams of the target radiotherapy plan; determining a first sorting result of the target radiotherapy plans based on the starting irradiation angle and the ending irradiation angle of each of the target radiotherapy plans; and generating the radiotherapy plan temporary list in which the target radiotherapy plans are sorted according to the first sorting result.

In some embodiments, the generating the radiotherapy plan temporary list based on the target radiotherapy plans includes for each of the target radiotherapy plans, determining irradiation angles of radiation beams of the target radiotherapy plan; determining a second sorting result of the radiation beams of the target radiotherapy plan based on the irradiation angles of the radiation beams of the target radiotherapy plan; and generating the radiotherapy plan temporary list in which the radiation beams of the target radiotherapy plan are sorted according to the second sorting result.

In some embodiments, the verifying the target radiotherapy plans in batches based on the radiotherapy plan temporary list using the at least one verification means of the at least one radiotherapy device includes controlling the at least one verification means to perform automatic positioning; and sequentially verifying the target radiotherapy plans in the radiotherapy plan temporary list, each of the target radiotherapy plans being verified using the verification means of one of the at least one radiotherapy device after automatic positioning.

In some embodiments, the sequentially verifying the target radiotherapy plans in the radiotherapy plan temporary list includes for each of the target radiotherapy plans in the radiotherapy plan temporary list, controlling the verification means that has experienced the automatic positioning to collect test data during an execution process of the target radiotherapy plan; determining whether the target radiotherapy plan meets a first preset condition based on the test data; and in response to determining that the target radiotherapy plan meets the first preset condition, verifying a next target radiotherapy plan in the radiotherapy plan temporary list; or in response to determining that the target radiotherapy plan does not meet the first preset condition, performing fault analysis on a corresponding radiotherapy device to obtain a fault analysis result.

In some embodiments, the performing fault analysis on the corresponding radiotherapy device to obtain the fault analysis result includes obtaining at least one device parameter of the radiotherapy device and generating the fault analysis result of the radiotherapy device based on the at least one device parameter.

In some embodiments, the generating the fault analysis result of the radiotherapy device based on the at least one device parameter includes generating the fault analysis result of the radiotherapy device by using a fault analysis model to process the at least one device parameter, the fault analysis model being a trained machine learning model.

In some embodiments, in response to determining that the target radiotherapy plan does not meet the first preset condition, the method further includes determining whether the radiotherapy device has a preset type of fault based on the fault analysis result and in response to determining that the radiotherapy device has the preset type of fault, terminating or skipping the verification of the target radiotherapy plan and verifying a next target radiotherapy plan in the radiotherapy plan temporary list.

In some embodiments, in the radiotherapy plan temporary list, the target radiotherapy plans are divided into radiotherapy plan groups based on at least one feature parameter of each of the target radiotherapy plans, and the verifying the target radiotherapy plans in batches includes for each of the radiotherapy plan groups, sequentially verifying one or more target radiotherapy plans in the radiotherapy plan group; whenever one of the one or more target radiotherapy plans in the radiotherapy plan group passes the verification, determining whether the radiotherapy plan group meets a second preset condition, and in response to determining that the radiotherapy plan group meets the second preset condition, determining that other target radiotherapy plans in the radiotherapy plan group pass the verification.

In some embodiments, the at least one feature parameter includes at least one of a radiotherapy type, a field location, or a field size.

In some embodiments, the at least one radiotherapy device includes multiple radiotherapy devices. The verifying the target radiotherapy plans in batches based on the radiotherapy plan temporary list through the at least one verification means of the at least one radiotherapy device may include determining reference information of each of the radiotherapy devices, the reference information including at least one of reservation information or historical usage information and assigning the target radiotherapy plans in the radiotherapy plan temporary list to the radiotherapy devices for verification based on the reference information of each of the radiotherapy devices.

In some embodiments, the at least one verification means of the at least one radiotherapy device includes at least one of an electronic portal imaging device (EPID) or a radiation detector.

Another aspect of the present disclosure relates to a system for image analysis. The system includes at least one storage device including a set of instructions and at least one processor in communication with the at least one storage device. When executing the set of instructions, the at least one processor is directed to cause the system to implement operations. The operations include obtaining target radiotherapy plans which need to be verified; generating a radiotherapy plan temporary list based on the target radiotherapy plans; and verifying the target radiotherapy plans in batches based on the radiotherapy plan temporary list using at least one verification means of at least one radiotherapy device.

A further aspect of the present disclosure relates to a non-transitory computer readable medium including executable instructions. When the executable instructions are executed by at least one processor, the executable instructions direct the at least one processor to perform a method. The method includes obtaining target radiotherapy plans which need to be verified; generating a radiotherapy plan temporary list based on the target radiotherapy plans; and verifying the target radiotherapy plans in batches based on the radiotherapy plan temporary list using at least one verification means of at least one radiotherapy device.

Additional features may be set forth in part in the description which follows, and in part may become apparent to those skilled in the art upon examination of the following and the accompanying drawings or may be learned by production or operation of the examples. Of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of example embodiments. These example embodiments are described in detail with reference to the drawings. The drawings are not to scale. These embodiments are non-limiting example embodiments, in which like reference numerals represent similar structures throughout the several views of the drawings, and wherein:

FIG. 1 a schematic diagram illustrating an exemplary radiotherapy plan verification system according to some embodiments of the present disclosure;

FIG. 2 is a block diagram illustrating an exemplary radiotherapy plan verification system according to some embodiments of the present disclosure;

FIG. 3 is a flowchart illustrating an exemplary process for radiotherapy plan verification according to some embodiments of the present disclosure;

FIG. 4 is a schematic diagram illustrating an exemplary process for generating a radiotherapy plan temporary list according to some embodiments of present disclosure;

FIG. 5 is a schematic diagram illustrating an exemplary process for generating a radiotherapy plan temporary list according to some embodiments of present disclosure;

FIG. 6 is a flowchart illustrating an exemplary process for verifying target radiotherapy plans in a radiotherapy plan temporary list according to some embodiments of the present disclosure;

FIG. 7 is a schematic diagram illustrating an exemplary process for verifying target radiotherapy plans in a radiotherapy plan temporary list according to some embodiments of the present disclosure;

FIG. 8 is a flowchart illustrating an exemplary process of radiotherapy plan verification according to some embodiments of the present disclosure;

FIG. 9 is a flowchart illustrating an exemplary process of radiotherapy plan verification according to some embodiments of present disclosure;

FIG. 10 is a schematic diagram illustrating an exemplary user interface for selecting target radiotherapy plans according to some embodiments of the present disclosure;

FIG. 11 is a schematic diagram illustrating an exemplary user interface for displaying a verification result of target radiotherapy plans according to some embodiments of the present disclosure; and

FIG. 12 is a schematic diagram illustrating an exemplary verification report of a target radiotherapy plan according to some embodiments of the present disclosure.

### DETAILED DESCRIPTION

In the following detailed description, numerous specific details may be set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the Disclosed embodiments may be readily apparent to those skilled in the art, and the general principles defined herein may be applied to other embodiments and applications without departing from the spirit and scope of the present disclosure. Thus, the present disclosure may be not limited to the embodiments shown, but to be consistent with the widest scope consistent with the claims.

The terminology used herein may be for the purpose of describing particular example embodiments only and may be not intended to be limiting. As used herein, the singular forms “a,” “an,” and “the” may be intended to include the plural forms as well, unless the context clearly indicates otherwise. It may be understood that the terms “system,” “unit,” “module,” and/or “block” used herein are one method to distinguish different components, elements, parts, sections, or assemblies of different levels in ascending order. However, the terms may be displaced by another expression if they achieve the same purpose.

The modules (or units, blocks, units) described in the present disclosure may be implemented as software and/or hardware modules and may be stored in any type of non-transitory computer-readable medium or other storage devices. In some embodiments, a software module may be compiled and linked into an executable program. It may be appreciated that software modules may be callable from other modules or from themselves, and/or may be invoked in response to detected events or interrupts. Software modules configured for execution on computing devices may be provided on a computer readable medium or as a digital download (and can be stored originally in a compressed or installable format that requires installation, decompression, or decryption prior to execution). Such software code may be stored, partially or fully, on a memory device of the executing computing device, for execution by the computing device. Software instructions may be embedded in a firmware, such as an EPROM. It may be further appreciated that hardware modules (e.g., circuits) may be included in connected or coupled logic units, such as gates and flip-flops, and/or may be included in programmable units, such as programmable gate arrays or processors. The modules or computing device described functionality herein may be preferably implemented as hardware modules, but may be software modules as well. In general, the modules described herein refer to logical modules that may be combined with other modules or divided into units despite their physical organization or storage.

Certain terminology has been used to describe embodiments of the present disclosure. For example, the terms “one embodiment,” “an embodiment,” and/or “some embodiments” may mean that a particular feature, structure, or characteristic described in connection with the embodiment is in at least one embodiment of the present disclosure. Therefore, it is emphasized and should be appreciated that two or more references to “an embodiment” or “one embodiment” or “an alternative embodiment” in various portions of this specification may not necessarily all referring to the same embodiment. Furthermore, the particular features, structures, or characteristics may be combined as suitable in one or more embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings may be for the purpose of illustration and description only and may be not intended to limit the scope of the present disclosure.

The flowcharts used in the present disclosure may illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

Before a radiotherapy plan is implemented, radiotherapy plan verification is usually required to ensure the accuracy of the treatment. Currently, radiotherapy plan verification usually relies on a third-party test tool or an electronic portal imaging device (EPID). However, both of the above modes of radiotherapy plan verification require a large amount of human resources. When using the third-party test tool for radiotherapy plan verification, a physiotherapist (i.e., a radiotherapy operator) needs to spend a lot of time on phantom positioning. In radiotherapy plan verification, the physiotherapist is also required to manually execute radiotherapy plans one by one and repeat a large number of mechanical operations. When using EPID for radiotherapy plan verification, the physiotherapist does not need to spend a lot of time on phantom positioning, but the physiotherapist still needs to manually load the radiotherapy plans and execute them one by one, which also takes a lot of time and energy of the physiotherapist.

In order to solve the above problems, the present disclosure provides methods and systems for radiotherapy plan verification in batches. The systems may determine target radiotherapy plans from a plurality of radiotherapy plans which need to be verified. The systems may generate a radiotherapy plan temporary list based on the target radiotherapy plans. Further, the systems may verify the target radiotherapy plans in batches based on the radiotherapy plan temporary list using verification means (e.g., the EPID) of at least one radiotherapy device.

According to the embodiments of the present disclosure, the radiotherapy plan temporary list is generated by selecting radiotherapy plans that need to be verified, and the radiotherapy plans that need to be verified are automatically verified in batches based on the radiotherapy plan temporary list. In the above process, no human participation or little human participation is required, which greatly reduces the workload of the radiotherapy operator (e.g., the physiotherapist) and solves the problem that a lot of manpower and time are needed to complete radiotherapy plan verification during radiotherapy (e.g., IMRT), thereby greatly improving the efficiency of radiotherapy.

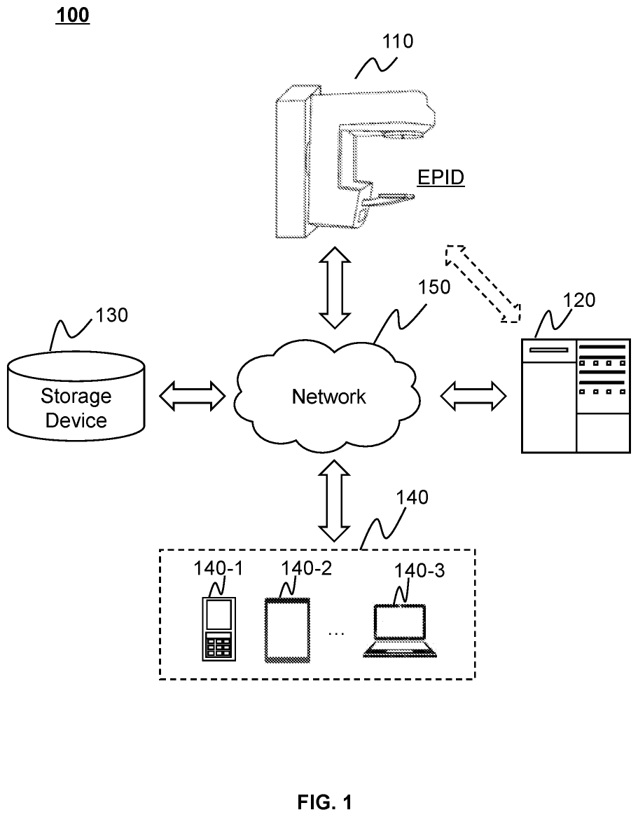

FIG. 1 is a schematic diagram illustrating an exemplary radiotherapy plan verification system 100 according to some embodiments of the present disclosure. As shown in FIG. 1, the radiotherapy plan verification system 100 may include a radiotherapy device 110, a processing device 120, a storage device 130, a terminal 140, and a network 150.

The radiotherapy device 110 refers to a medical device that uses radiation to treat patients. In some embodiments, the radiotherapy device 110 may be any medical device capable of treating a designated body part of a patient through radiation (e.g., gamma rays, electron rays, neutron rays, etc.).

In some embodiments, the radiotherapy device 110 may include a verification means. The verification means may include at least one of an electronic portal imaging device (EPID), or radiation detector. The verification means may be controlled to perform automatic positioning, and radiotherapy plans may be verified using the verification means after automatic positioning. In some embodiments, the radiotherapy device 110 may receive an instruction sent by a radiotherapy operator (e.g., a physiotherapist, etc.) via the terminal 140, and perform, based on the instruction, a relevant operation, such as executing a radiotherapy plan. In some embodiments, the radiotherapy device 110 may perform data and/or information exchange with other components (e.g., the processing device 120, the storage device 130, and the terminal 140) of the radiotherapy plan verification system 100 through the network 150. In some embodiments, the radiotherapy device 110 may be directly connected with other components of the radiotherapy plan verification system 100. In some embodiments, one or more components (e.g., the processing device 120, and the storage device 130) of the radiotherapy plan verification system 100 may be integrated into the radiotherapy device 110.

The processing device 120 may process data and/or information obtained from other devices or components, and perform radiotherapy plan verification methods shown in the embodiments of the present disclosure. For example, the processing device 120 may generate a radiotherapy plan temporary list containing target radiotherapy plans based on a user instruction of the terminal 140. As another example, the processing device 120 may control the verification means of the radiotherapy device 110 to perform automatic positioning. In some embodiments, the processing device 120 may verify the target radiotherapy plans in batches in the radiotherapy plan temporary list. In some embodiments, the processing device 120 may obtain, from the storage device 130, pre-stored data and/or information, such as the radiotherapy plans, the user instruction, etc., to execute the radiotherapy plan verification method in some embodiments of the present disclosure.

In some embodiments, the processing device 120 may include one or more sub-processing devices (e.g., a single-core processing device or a multi-core processing device). Merely by way of example, the processing device 120 may include a central processing unit (CPU), an application specific integrated circuit (ASIC), an application specific instruction processor (ASIP), a graphics processing unit (GPU), a physical processor (PPU), a digital signal processor (DSP), a field programmable gate array (FPGA), a programmable logic circuit (PLD), a controller, a microcontroller unit, a reduced instruction set computer (RISC), a microprocessor, or the like, or any combination thereof.

The storage device 130 may store data or information generated by other devices. In some embodiments, the storage device 130 may store various information and/or data, e.g., the radiotherapy plans of the at least one radiotherapy device 110, one or more filtering conditions of the radiotherapy plans, the radiotherapy plan temporary list, a fault analysis model, etc. The storage device 130 may include one or more storage components. Each storage component may be an independent device or a part of another device. The storage device may be local or implemented by a cloud computing platform.

The terminal 140 may control operations of the radiotherapy device 110 and/or the processing device 120. The radiotherapy operator may issue an operation instruction to the radiotherapy device 110 through the terminal 140, so that the radiotherapy device 110 may complete a specified operation, e.g., positioning, executing a radiotherapy plan, etc. In some embodiments, the terminal 140 may cause, through an instruction, the processing device 120 to execute the radiotherapy plan verification method in some embodiments of the present disclosure. In some embodiments, the terminal 140 may present radiotherapy plans. The radiotherapy operator may select target radiotherapy plans that need to be verified from the radiotherapy plans. In some embodiments, the terminal 140 may include a mobile device 140-1, a tablet computer 140-2, a laptop computer 140-3, a desktop computer, or any other device with input and/or output functions, or any combination thereof.

The network 150 may connect the components of the radiotherapy plan verification system 100 and/or connect the radiotherapy plan verification system 100 to external resources. The network 150 enables communication between the components of the radiotherapy plan verification system 100 and between the radiotherapy plan verification system 100 and other external components to facilitate data and/or information exchange. In some embodiments, one or more components (e.g., the radiotherapy device 110, the processing device 120, the storage device 130, and the terminal 140) of the system radiotherapy plan verification 100 may send data and/or information to other components via the network 150. In some embodiments, network 150 may be any one or more of a wired network or a wireless network.

It should be noted that the above description is provided for illustrative purposes only and is not intended to limit the scope of the present disclosure. For those having ordinary skills in the art, various changes and modifications may be made under the guidance of the contents of the present disclosure. The features, structures, methods, and other characteristics of the exemplary embodiments described in the present disclosure may be combined in various ways to obtain additional and/or alternative exemplary embodiments. For example, the processing device 120 may be implemented by a cloud computing platform, e.g., a public cloud, a private cloud, a community cloud, a hybrid cloud, etc. As another example, the radiotherapy plan verification system 100 may include multiple radiotherapy devices 110. However, such changes and modifications do not depart from the scope of the present disclosure.

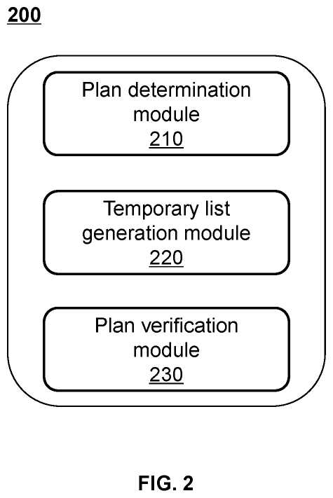

FIG. 2 is a block diagram illustrating an exemplary radiotherapy plan verification system 200 according to some embodiments of the present disclosure. As shown in FIG. 2, the radiotherapy plan verification system 200 may include a plan determination module 210, a temporary list generation module 220, and a plan verification module 230. In some embodiments, the radiotherapy plan verification system 200 may be implemented on the processing device 120.

The plan determination module 210 may be configured to determine target radiotherapy plans from a plurality of radiotherapy plans. More descriptions of the determination of the target radiotherapy plans may be found elsewhere in the present disclosure (e.g., operation 310 and the descriptions thereof).

The temporary list generation module 220 may be configured to generate a radiotherapy plan temporary list based on the target radiotherapy plans. More descriptions of the generation of the radiotherapy plan temporary list may be found elsewhere in the present disclosure (e.g., operation 320 and the descriptions thereof).

The plan verification module 230 plan verification module 230 may be configured to verify the target radiotherapy plans in batches based on the radiotherapy plan temporary list using at least one verification means of the at least one radiotherapy device. More descriptions of the verification of the target radiotherapy plans may be found elsewhere in the present disclosure (e.g., operation 330 and the descriptions thereof).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the radiotherapy plan verification system 200 may include one or more additional modules, such as a storage module (not shown) for storing data.

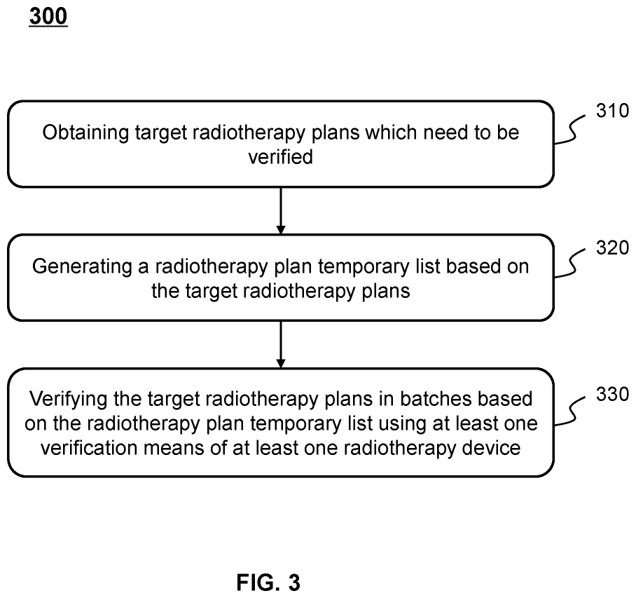

FIG. 3 is a flowchart illustrating an exemplary process 300 of radiotherapy plan verification according to some embodiments of the present disclosure. As shown in FIG. 3, the process 300 may include the following operations. In some embodiments, the process 300 may be implemented by the radiotherapy plan verification system 100 (e.g., the processing device 120) or the radiotherapy plan verification system 200 (e.g., one or more modules illustrated in FIG. 2).

In 310, target radiotherapy plans may be determined from a plurality of radiotherapy plans. The target radiotherapy plans need to be verified. In some embodiments, operation 310 may be implemented by the plan determination module 210 illustrated in FIG. 2.

A radiotherapy plan may be used to define how a radiotherapy is performed on a treatment subject (e.g., a patient, etc.). For example, the radiotherapy plan may include a total radiation dose of the radiotherapy, a dose in each treatment fraction of the radiotherapy, an angle and a shape of a radiation beam in each treatment fraction of the radiotherapy, etc. In radiotherapy, especially IMRT, in order to ensure that the radiation can accurately irradiate the lesion of the treatment subject, it is particularly important to verify the radiotherapy plan before the delivery of the radiotherapy.

The target radiotherapy plan refers to a radiotherapy plan that needs to be verified. In some embodiments, radiotherapy plan verification refers to collecting test data during the execution of the radiotherapy plan and determining whether the radiotherapy plan meets the treatment expectations and requirements (e.g., whether the radiotherapy can be delivered precisely according to the radiotherapy plan) based on the test data. For example, dose information (e.g., dose distribution information, etc.) may be obtained through a verification means, and whether the radiotherapy plan meets the treatment requirements may be determined by comparing the obtained dose information with planned dose information. In some embodiments, during the execution of the radiotherapy plan, the verification means may directly obtain grayscale value information. Since a grayscale value and a dose are linearly related, the obtained grayscale value information may be converted into the dose information.

The plan determination module 210 may determine the target radiotherapy plans from the radiotherapy plans in various ways. In some embodiments, the plan determination module 210 may designate all the radiotherapy plans as the target radiotherapy plans.

In some embodiments, a user may select radiotherapy plans to be verified from the radiotherapy plans as the target radiotherapy plans. Specifically, the plan determination module 210 may present, a user terminal (e.g., the terminal 140), the plurality of radiotherapy plans. For example, the plan determination module 210 may direct the user terminal to present the radiotherapy plans to the user (e.g., the radiotherapy operator) in the form of a list, an icon, or the like. Further, the plan determination module 210 may receive, via the user terminal, a user instruction for selecting the target radiotherapy plans from the radiotherapy plans. The user instruction refers to a computer instruction for selecting the target radiotherapy plans from the radiotherapy plans. Merely by way of example, the user may input the user instruction by a touch screen, a keyboard, a mouse, voice, etc.

In some embodiments, the user instruction may be used to select all or part of the radiotherapy plans as the target radiotherapy plans.

In some embodiments, the user instruction may be used to select the target radiotherapy plans based on one or more filtering conditions, i.e., the radiotherapy plans that meet the one or more filtering conditions may be selected as the target radiotherapy plans. For example, the plan determination module 210 may further direct the user terminal to present a filtering element for filtering the radiotherapy plans using the one or more filtering conditions, and the user instruction is input vis the filtering element presented by the user terminal. The one or more filtering conditions may include at least one of a treatment part, a treatment subject, a treatment type, etc. For example, the filtering conditions may be that the treatment part is the stomach and the treatment type is IMRT. As another example, the filtering conditions may be that the treatment part is the lungs, and the treatment subject is Zhang. The plan determination module 210 may select the radiotherapy plans that meet the filtering conditions from the radiotherapy plans as the target radiotherapy plans.

In some embodiments, the plan determination module 210 may automatically select the target radiotherapy plans from the radiotherapy plans. For example, the plan determination module 210 may select a plurality of radiotherapy plans whose planned delivery times are close to the current time as the target radiotherapy plans. As another example, the plan determination module 210 may select a plurality of newly made radiotherapy plans as the target radiotherapy plans.

As another example, the plan determination module 210 may obtain status information of at least one radiotherapy device (e.g., the at least one radiotherapy device 110). The at least one radiotherapy device may be used to verify the target radiotherapy plans. The status information of a radiotherapy device may include device parameters of the radiotherapy device, e.g., beam deviation of a multi-leaf collimator (MLC) of the radiotherapy device. The status information may be obtained by detecting or testing the operation status of the radiotherapy device.

Further, for each of the radiotherapy plans, the plan determination module 210 may determine planned radiotherapy parameters of the radiotherapy plan, and determine a risk coefficient of the radiotherapy plan based on the status information and the planned radiotherapy parameters. The planned radiotherapy parameters may include a beam angle, a beam dose, a beam shape, or the like. The plan determination module 210 may directly obtain the planned radiotherapy parameters from the corresponding radiotherapy plan. The risk coefficient may reflect a risk (possibility) that the corresponding radiotherapy plan does not pass subsequent verification.

In some embodiments, the plan determination module 210 may determine the risk coefficient of the radiotherapy plan by using a risk assessment model to process the status information and the planned radiotherapy parameters. The risk assessment model may be a pre-trained machine learning model. An input of the risk assessment model may include the status information and planned radiotherapy parameters of a radiotherapy plan, and an output of the risk assessment model may include the risk coefficient of the radiotherapy plan. Training samples used to train the risk assessment model may be determined based on historical radiotherapy verification records. A historical radiotherapy verification record may include historical planned radiotherapy parameters of a historical radiotherapy plan, historical status information of a corresponding radiotherapy device that was used to verify the historical radiotherapy plan, and a plan verification result of the historical radiotherapy plan. For example, the plan verification result may be represented by a value of 1 if the historical radiotherapy plan passes the verification, and represented by a value of 0 if the historical radiotherapy plan does not pass the verification. During a training process of the risk assessment model, historical planned radiotherapy parameters of a historical radiotherapy plan and historical status information of a corresponding radiotherapy device used to verify the historical radiotherapy plan may be used as a training input, and a plan verification result of the historical radiotherapy plan may be used as a training gold standard.

Further, the plan determination module 210 may select the target radiotherapy plans from the radiotherapy plans based on the risk coefficients of the radiotherapy plans. For example, the plan determination module 210 may select radiotherapy plans whose risk coefficients are relatively high or exceed a threshold as the target radiotherapy plans. According to the above embodiments, by performing a risk assessment on the radiotherapy plans, and then selecting the radiotherapy plans with a relatively high risk as the target radiotherapy plans for verification, a probability that the radiotherapy plans with a relatively high risk are omitted during the selection process of the target radiotherapy plans can be reduced, and the selection accuracy of the target radiotherapy plans can be improved.

In 320, a radiotherapy plan temporary list may be generated based on the target radiotherapy plans. In some embodiments, operation 320 may be implemented by the temporary list generation module 220 illustrated in FIG. 2.

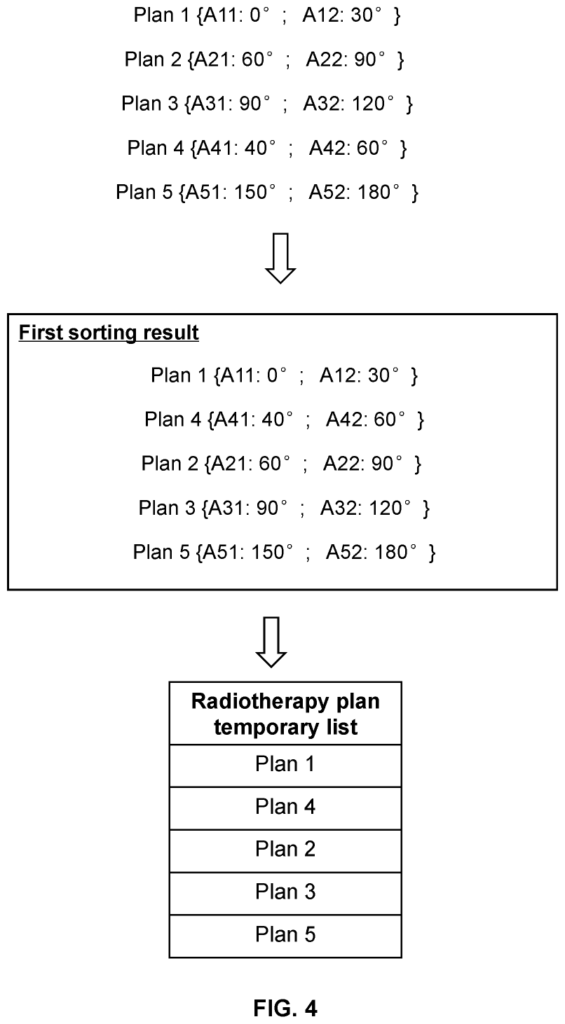

The radiotherapy plan temporary list refers to a temporary record table that stores information related to the target radiotherapy plans. In some embodiments, the target radiotherapy plans may be arranged in any order in the radiotherapy plan temporary list. In some embodiments, to improve verification efficiency, the target radiotherapy plans in the radiotherapy plan temporary list may be sorted in a specific order. For example, the target radiotherapy plans may be sorted based on the planned delivery time of each target radiotherapy plan. As another example, the target radiotherapy plans may be sorted based on irradiation angles of radiation beams of each radiotherapy plan. Specifically, for each of the target radiotherapy plans, the temporary list generation module 220 may determine a starting irradiation angle and an ending irradiation angle of the radiation beams of the target radiotherapy plan. The temporary list generation module 220 may determine a first sorting result of the target radiotherapy plans based on the starting irradiation angle and the ending irradiation angle of the radiation beams of the target radiotherapy plan. Further, the temporary list generation module 220 may generate the radiotherapy plan temporary list in which the target radiotherapy plans are sorted according to the first sorting result. More descriptions of the generation of the radiotherapy plan temporary list may be found elsewhere in the present disclosure (e.g., FIG. 4 and the descriptions thereof).

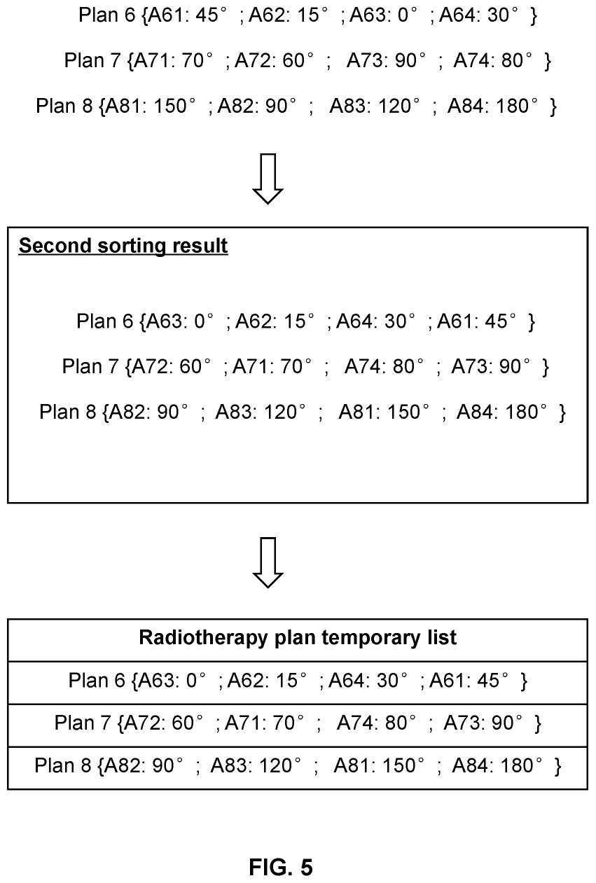

In some embodiments, in the radiotherapy plan temporary list, the radiation beams of each target radiotherapy plan may be sorted in a specific order. Specifically, for each of the target radiotherapy plans, the temporary list generation module 220 may determine irradiation angles of radiation beams of the target radiotherapy plan and determine a second sorting result of the radiation beams of the target radiotherapy plan based on the irradiation angles of the radiation beams of the target radiotherapy plan. Further, the temporary list generation module 220 may generate the radiotherapy plane temporary list in which the radiation beams of the target radiotherapy plan are sorted according to the second sorting result. More descriptions of the generation of the radiotherapy plan temporary list may be found elsewhere in the present disclosure (e.g., FIG. 5 and the descriptions thereof).

In some embodiments, the radiotherapy plan temporary list may be temporarily stored and automatically deleted after all the radiotherapy plans in the radiotherapy plan temporary list are verified. In some embodiments, the radiotherapy plan temporary list may be stored in a storage device (e.g., the storage device 130, a cache device) for later use.

In 330, the target radiotherapy plans may be verified in batches based on the radiotherapy plan temporary list using at least one verification means of the at least one radiotherapy device. In some embodiments, operation 330 may be implemented by the plan verification module 230 illustrated in FIG. 2.

As used herein, verifying the target radiotherapy plan in batches refers to that the target radiotherapy plans are verified at the same time or in sequence without user intervention or with little user intervention.

In some embodiments, the plan verification module 230 may control the at least one verification means of the at least one radiotherapy device to perform automatic positioning. Automatic positioning refers to driving a verification means to a preset position through an electric device (e.g., a motor). For example, after the user inputs an instruction to start the verification of the target radiotherapy plans, the plan verification module 230 may control the verification means of each radiotherapy device to move to a measurement position. In such cases, the verification means may be aligned with a radioactive source (e.g., a laser light, etc.) of the corresponding radiotherapy device without additional manual intervention, thereby saving manpower and time required for positioning.

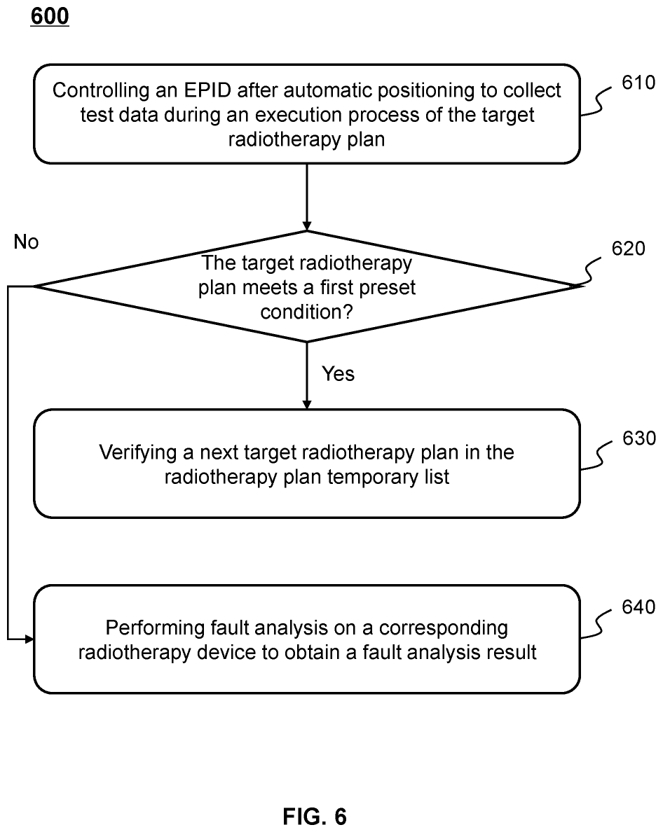

Further, the plan verification module 230 may sequentially verify the target radiotherapy plans in the radiotherapy plan temporary list, wherein each target radiotherapy plan may be verified using the verification means of one radiotherapy device after automatic positioning. Specifically, for each of the target radiotherapy plans in the radiotherapy plan temporary list, the plan verification module 230 may control a verification means of one radiotherapy device after automatic positioning to collect test data during an execution process of the target radiotherapy plan. Further, the plan verification module 230 may determine whether the target radiotherapy plan meets a first preset condition based on the test data. In response to determining that the target radiotherapy plan meets the first preset condition, the plan verification module 230 may verify a next target radiotherapy plan in the radiotherapy plan temporary list. In response to determining that the target radiotherapy plan does not meet the first preset condition, the plan verification module 230 may perform fault analysis on a corresponding radiotherapy device to obtain a fault analysis result. More descriptions of the verification of the target radiotherapy plans and the failure analysis may be found elsewhere in the present disclosure (e.g., FIG. 6 and the descriptions thereof).

In some embodiments, for each of the target radiotherapy plans in the radiotherapy plan temporary list, the plan verification module 230 may obtain a radiation dose to adjacent organs of a target region (e.g., a lesion) during the execution process of the target radiotherapy plan. Further, the plan verification module 230 may determine whether the radiation dose to the adjacent organs of the target region exceeds a maximum dose threshold. If the radiation dose to the adjacent organs of the target region does not exceed the maximum dose threshold, the plan verification module 230 may verify a next target radiotherapy plan in the radiotherapy plan temporary list. If the radiation dose to the adjacent organs of the target region exceeds the maximum dose threshold, the plan verification module 230 may send a notification to a user and/or perform the fault analysis on the corresponding radiotherapy device to obtain a fault analysis result.

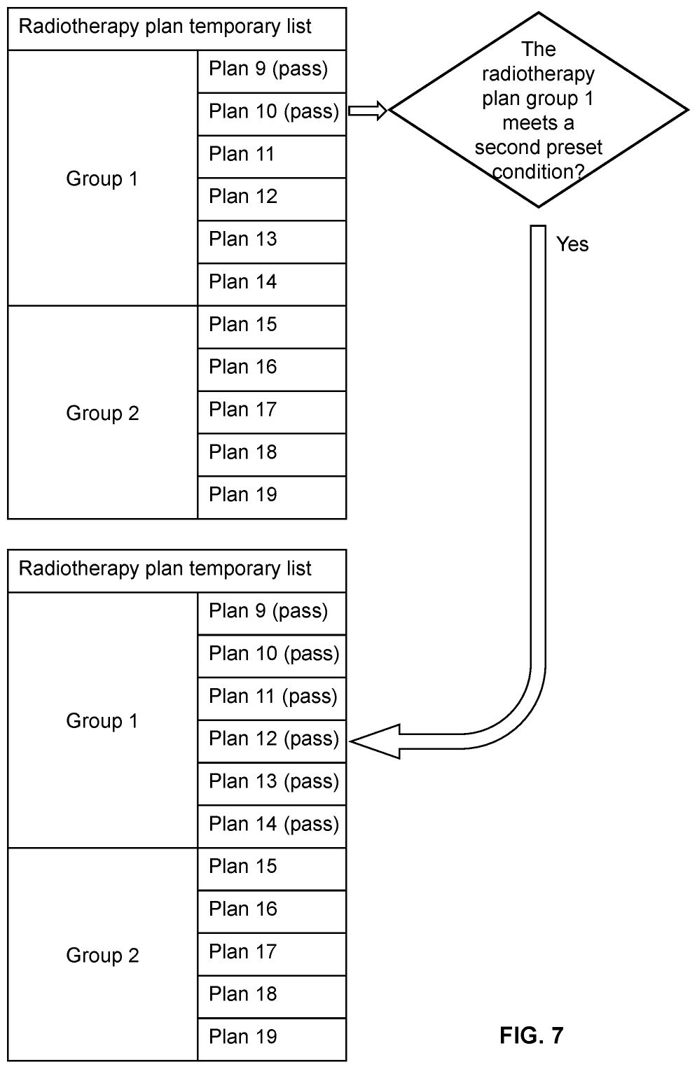

In some embodiments, the plan verification module 230 may perform group verification on the target radiotherapy plans in the radiotherapy plan temporary list. Specifically, in the radiotherapy plan temporary list, the target radiotherapy plans may be divided into radiotherapy plan groups based on at least one feature parameter of each of the target radiotherapy plans. Merely by way of example, the at least one feature parameter may include at least one of a radiotherapy type, a field location, or a field size. The radiotherapy type may include dynamic IMRI and/or static IMRI. The field size refers to a size of a region irradiated by the radiation beams (or referred to as an irradiation region). The field location refers to a location of the region irradiated by the radiation beams (e.g., a location of a center point of the region). The field location may include a distance between the location of the center point of the region irradiated by the radiation beams and a center of the target region. Further, for each of the radiotherapy plan groups, the plan verification module 230 may sequentially verify one or more target radiotherapy plans in the radiotherapy plan group. Whenever one of the one or more target radiotherapy plans in the radiotherapy plan group passes the verification, the plan verification module 230 may determine whether the radiotherapy plan group meets a second preset condition. In response to determining that the radiotherapy plan group meets the second preset condition, the plan verification module 230 may determine that other target radiotherapy plans in the radiotherapy plan group may pass the verification. More descriptions of the group verification may be found elsewhere in the present disclosure (e.g., FIG. 7 and the descriptions thereof).

In some embodiments, the at least one radiotherapy device may include multiple radiotherapy devices, e.g., radiotherapy devices capable of verifying the target radiotherapy plans. The plan verification module 230 may determine reference information of each of the radiotherapy devices. Merely by way of example, the reference information may include at least one of reservation information and historical usage information. The reservation information may include information such as a count of reservations of each radiotherapy device on the day of verifying the target radiotherapy plans, a time period of each of the reservations, or the like. The historical usage information may include information such as installation time, a historical usage count, and a historical usage frequency of each radiotherapy device.

Further, the plan verification module 230 may assign the target radiotherapy plans in the radiotherapy plan temporary list to the radiotherapy devices for verification based on the reference information of each of the radiotherapy devices. For example, the plan verification module 230 may assign more target radiotherapy plans to a radiotherapy device that has a small count of reservations and a relatively concentrated unreserved time period, to avoid affecting the use of the radiotherapy device for normal treatment. As another example, the plan verification module 230 may assign more target radiotherapy plans to a radiotherapy device that has an early installation time (i.e., an old machine), a large historical usage count, or a high historical usage frequency, to reduce wear and tear on new radiotherapy devices.

In some embodiments, the plan verification module 230 may assign target radiotherapy plans of a same radiotherapy plan group to a same radiotherapy device for verification. In some embodiments, when the reservation information of one or more radiotherapy devices of the radiotherapy devices is adjusted, the plan verification module 230 needs to adjust an assignment result of the target radiotherapy plans in real time. According to the above embodiments, the radiotherapy devices may simultaneously verify the target radiotherapy plans, thereby improving the utilization rate of the radiotherapy devices and the verification efficiency of the target radiotherapy plans.

According to the embodiments of the present disclosure, the radiotherapy plan temporary list is generated by selecting the radiotherapy plans that need to be verified, and the radiotherapy plans that need to be verified are automatically verified in batches based on the radiotherapy plan temporary list. In the above process, no human participation or little human participation is required, which greatly reduces the workload of the radiotherapy operator (e.g., the physiotherapist) and solves the problem that a lot of manpower and time are needed to complete radiotherapy plan verification during radiotherapy (e.g., IMRT), thereby greatly improving the efficiency of radiotherapy.

FIG. 4 is a schematic diagram illustrating an exemplary process for generating a radiotherapy plan temporary list according to some embodiments of the present disclosure. Each target radiotherapy plan may include a plurality of radiation beams with different irradiation angles emitted in sequence. When a target radiotherapy plan is verified, a gantry of the radiotherapy device needs to rotate to different irradiation angles for irradiating the radiation beams. The target radiotherapy plan may include a plurality of gantry angles (i.e., irradiation angles) each of which corresponds to one radiation beam.

The irradiation angles may include a starting irradiation angle and an ending irradiation angle. The starting irradiation angle refers to an irradiation angle of a first radiation beam planned to be emitted in a target radiotherapy plan. The ending irradiation angle refers to an irradiation angle of a last radiation beam planned to be emitted in the target radiotherapy plan. As shown in FIG. 4, a starting irradiation angle A11 and an ending irradiation angle A12 of radiation beams of a target radiotherapy plan 1 may be 0 degrees and 30 degrees, respectively; a starting irradiation angle A21 and an ending irradiation angle A22 of radiation beams of a target radiotherapy plan 2 may be 60 degrees and 90 degrees, respectively; a starting irradiation angle A31 and an ending irradiation angle A32 of radiation beams of a target radiotherapy plan 3 may be 90 degrees and 120 degrees, respectively; a starting irradiation angle A41 and an ending irradiation angle A42 of radiation beams of a target radiotherapy plan 4 may be 40 degrees and 60 degrees, respectively; and a starting irradiation angle A51 and an ending irradiation angle A52 of radiation beams of a target radiotherapy plan 5 may be 150 degrees and 180 degrees, respectively.

The temporary list generation module 220 may determine the starting irradiation angle and the ending irradiation angle of the radiation beams of each target radiotherapy plan, and determine a first sorting result of the target radiotherapy plans based on the starting irradiation angle and the ending irradiation angle of the radiation beams of each target radiotherapy plan. In some embodiments, as shown in FIG. 4, in the first sorting result, a starting irradiation angle of a latter target radiotherapy plan may be close to an ending irradiation angle of a previous target radiotherapy plan. For example, the target radiotherapy plan 4 is arranged immediately after the target radiotherapy plan 1 in the first storing result. The ending irradiation angle of the target radiotherapy plan 1 may be 30 degrees (i.e., a gantry angle is 30 degrees after plan 1 is verified), and the starting irradiation angle of the latter target radiotherapy plan 4 may be 40 degrees (i.e., an angle of the gantry at the start of plan 4 verification is 40 degrees). In this way, after the verification of plan 1 is ended, the gantry only needs to rotate a relatively small angle to verify plan 4, thereby improving the verification efficiency of the target radiotherapy plan.

In some embodiments, the temporary list generation module 220 may determine a plurality of possible sorting results by randomly sorting the target radiotherapy plans. For each of the possible sorting results, the temporary list generation module 220 may determine a plurality of pairs of adjacent radiotherapy plans in the sorting result. The temporary list generation module 220 may determine an absolute value of a difference between the ending irradiation angle of the previous target radiotherapy plan and the starting irradiation angle of the latter target radiotherapy plan in each pair of adjacent radiotherapy plans, and then determine a sum of the absolute values of the differences of the plurality of pairs of adjacent radiotherapy plans. The temporary list generation module 220 may select a sorting result corresponding to a smallest sum of the absolute values of the differences from the plurality of possible sorting results as the first sorting result. Further, as shown in FIG. 4, the temporary list generation module 220 may generate the radiotherapy plan temporary list in which the target radiotherapy plans are sorted according to the first sorting result.

In some embodiments, for each of the target radiotherapy plans, the temporary list generation module 220 may determine irradiation angles of radiation beams of the target radiotherapy plan and determine a second sorting result of the radiation beams of the target radiotherapy plan based on the irradiation angles of the radiation beams. For example, as shown in FIG. 5, irradiation angles A61, A62, A63, and A64 of four radiation beams of a target radiotherapy plan 6 may be 45 degrees, 15 degrees, 0 degree, and 30 degrees, respectively; irradiation angles A71, A72, A73, and A74 of four radiation beams of a target radiotherapy plan 7 may be 70 degrees, 60 degrees, 90 degrees, and 80 degrees, respectively; and irradiation angles A81, A82, A83, and A84 of four radiation beams of a target radiotherapy plan 8 may be 150 degrees, 90 degrees, 120 degrees, and 180 degrees, respectively.

In some embodiments, the temporary list generation module 220 may determine the second sorting result of the radiation beams by sorting the irradiation angles of the radiation beams of the target radiotherapy plan in an ascending order. For example, for the target radiotherapy plan 6, the temporary list generation module 220 may determine the second sorting result of the radiation beams of the target radiotherapy plan 6 by sorting the irradiation angles A61, A62, A63, and A64 in an ascending order; for the target radiotherapy plan 7, the temporary list generation module 220 may determine the second sorting result of the radiation beams of the target radiotherapy plan 7 by sorting the irradiation angles A71, A72, A73, and A74 in an ascending order; and for the target radiotherapy plan 8, the temporary list generation module 220 may determine the second sorting result of the radiation beams of the target radiotherapy plan 8 by sorting the irradiation angles A81, A82, A83, and A84 in an ascending order.

Further, the temporary list generation module 220 may generate the radiotherapy plan temporary list in which the radiation beams of each target radiotherapy plan in the radiotherapy plan temporary list are sorted according to second sorting results corresponding to the target radiotherapy plan. In this embodiment, for example, the irradiation angles of the four radiation beams of the target radiotherapy plan 6 are 45 degrees, 15 degrees, 0 degrees, and 30 degrees, respectively. If the target radiotherapy plan 6 is verified when the irradiation angles of the four radiation beams of the target radiotherapy plan 6 are not sorted, the gantry of the radiotherapy device needs to be rotated from 45 degrees to 15 degrees, then to 0 degrees, and then to 30 degrees. If the target radiotherapy plan 6 is verified when the irradiation angles of the four radiation beams of the target radiotherapy plan 6 are sorted, the gantry of the radiotherapy device may be rotated from 0 degrees to 15 degrees, then to 30 degrees, and then to 40 degrees. Obviously, compared with the verification process of the target radiotherapy plan 6 when the irradiation angles are not sorted, during the verification process of the target radiotherapy plan 6 when the irradiation angles are sorted, the angle of gantry rotation becomes smaller, thereby improving the verification efficiency.

In some embodiments, after the irradiation angles of the radiation beams of each target radiotherapy plan are sorted, the target radiotherapy plans may be stored randomly or based on a specific rule to generate the radiotherapy plan temporary list. For example, the temporary list generation module 220 may sort the target radiotherapy plans in the radiotherapy plan temporary list based on planned delivery time of each target radiotherapy plan. As another example, after the irradiation angles of the radiation beams of each target radiotherapy plan are sorted, the temporary list generation module 220 may determine the first sorting result of all the target radiotherapy plans based on the starting irradiation angle and the ending irradiation angle of each of the target radiotherapy plans, and sort all the target radiotherapy plans according to the first sorting result in the generated radiotherapy plan temporary list. It should be noted that in this case, the starting irradiation angle and the ending irradiation angle of the radiation beams of each target radiotherapy plan may be a minimum irradiation angle and a maximum irradiation angle of the target radiotherapy plan in the second sorting result, respectively.

FIG. 6 is a flowchart illustrating an exemplary process 600 for verifying target radiotherapy plans in a radiotherapy plan temporary list according to some embodiments of the present disclosure. As shown in FIG. 6, the process 600 may include the following operations. In some embodiments, the process 600 may be implemented by the processing device 120 or the plan verification module 230. In some embodiments, at least part of operation 330 described in FIG. 3 may be implemented by executing the operations in the process 600. In some embodiments, the operations in the process 600 may be implemented for each target radiotherapy plan in the radiotherapy plan temporary list.

In 610, a verification means after automatic positioning may be controlled to collect test data during an execution process of a target radiotherapy plan.

In some embodiments, the plan verification module 230 may control a radiotherapy device to implement the target radiotherapy plan. Specifically, the plan verification module 230 may control a radioactive source of the radiotherapy device to emit radiation beams according to the target radiotherapy plan in the radiotherapy plan temporary list. During the execution process of the target radiotherapy plan, the plan verification module 230 may control the verification means of the radiotherapy device after automatic positioning to receive the radiation beams emitted by the radioactive source, thereby collecting the test data. The test data may include data that reflects a radiation receiving condition, e.g., a radiation photon energy value, a radiation photon count, etc. In some embodiments, the test data may be collected for each radiation beam. After the target radiotherapy plan is executed, the plan verification module 230 may synthesize all the collected test data of each radiation beam to obtain the test data of the target radiotherapy plan.

In 620, whether the target radiotherapy plan meets a first preset condition may be determined based on the test data.

In some embodiments, the plan verification module 230 may determine whether the target radiotherapy plan meets the first preset condition by comparing the currently obtained test data and theoretical data. The theoretical data may correspond to the test data. For example, if the test data is radiation photon count that the verification means actually receives, the theoretical data may be theoretical radiation photon count that the verification means receives. For example, the plan verification module 230 may determine whether a difference between the theoretical data and the test data exceeds a first preset threshold. The first preset threshold may be preset by the system. If the difference between the theoretical data and the test data does not exceed the first preset threshold, the plan verification module 230 may determine that the target radiotherapy plan meets the first preset condition. If the difference between the theoretical data and the test data exceeds the first preset threshold, the plan verification module 230 may determine that the target radiotherapy plan does not meet the first preset condition.

In some embodiments, the plan verification module 230 may determine a gamma pass rate based on the test data and the theoretical data. The plan verification module 230 may determine whether the gamma pass rate reaches or exceeds a second preset threshold. The second preset threshold may be set according to a disease type, a treatment type, objective needs, experience, etc. For example, for an IMRT plan of cervical cancer, under the standards of 2 mm/2% and 3 mm/3%, the second preset threshold may be set to 90% or 95%. As used herein, 2 mm/2% and 3 mm/3% refers to gamma pass rate standards. 3 mm or 2 mm represents a distance (i.e., distance to agreement, referred to as DTA) between a measurement point and a calculated point with the same dose. 3% or 2% represents a dose difference (referred to as DD) between a calculated value and a measured value at the same position. If the gamma pass rate reaches or exceeds the second preset threshold, the plan verification module 230 may determine that the target radiotherapy plan meets the first preset condition. If the gamma pass rate does not reach the second preset threshold, the plan verification module 230 may determine that the target radiotherapy plan does not meet the first preset condition.

When the target radiotherapy plan meets the first preset condition, it is considered that the target radiotherapy plan passes the verification, and the plan verification module 230 may perform operation 630. When the target radiotherapy plan does not meet the first preset condition, it is considered that the target radiotherapy plan does not pass the verification, and the plan verification module 230 may perform operation 640.

In 630, a next target radiotherapy plan in the radiotherapy plan temporary list may be verified.

For the next target radiotherapy plan, the plan verification module 230 may perform operations 610 and 620 again to verify the target radiotherapy plan.

In 640, fault analysis may be performed on the radiotherapy device to obtain a fault analysis result.

The fault analysis result may include descriptions of a fault, a fault type, an analysis of a fault cause, etc. In some embodiments, the fault analysis may be performed in various ways, e.g., machine QA (Quality Assessment) of the radiotherapy device, machine learning model processing, manual analysis, etc.

For example, the plan verification module 230 may control the radiotherapy device to perform machine QA to automatically generate the fault analysis result (e.g., a fault cause analysis report). Machine QA of the radiotherapy device may include beam QA, MLC QA, etc.

As another example, the plan verification module 230 may obtain at least one device parameter of the radiotherapy device. The plan verification module 230 may obtain the at least one device parameter of the radiotherapy device in various ways (e.g., the machine QA, etc.). The device parameter may include types (e.g., X-rays, electrons, protons, heavy ions, etc.), energy (e.g., 6MV, 10MV, etc.), radiation fields (e.g., 30*30 cm, 40*40 cm, etc.) of radiation beams, a treatment type (e.g., stereotactic radiosurgery (SRS), IMRT, stereotactic body radiation therapy (SBRT), etc.), a position and/or angle of each component of the radiotherapy device, etc. Further, the plan verification module 230 may generate the fault analysis result of the radiotherapy device based on the at least one device parameter. For example, the plan verification module 230 may generate the fault analysis result of the radiotherapy device by using a fault analysis model to process the at least one device parameter. The fault test model may be a trained machine learning model, e.g., a decision tree model, a neural network model, etc. In some embodiments, an input of the fault test model may include the obtained at least one device parameter, and an output of the fault test model may include the fault analysis result (e.g., a fault type, a fault cause). In some embodiments, a training sample used to train the fault test model may include device parameters, and a label used to train the fault test model may include a manually labeled fault analysis result (e.g., manually labeled fault type, manually labeled fault cause).

In some embodiments, the plan verification module 230 may determine whether the radiotherapy device has a preset type of failure based on the fault analysis result. The preset type of failure may not have a substantial impact on the implementation of the radiotherapy plan, which means that the preset type of failure is a minor failure that can be ignored. The preset type of failure refers to a failure that has been evaluated by a system engineer and would not cause damage to the radiotherapy device. For example, when executing the radiotherapy plan for a next patient, the previous patient must be unlocked first before the next patient can be executed, the preset type of failure may include that the previous patient not being unlocked. The preset type of failure may also include the inability to load a next radiotherapy plan and/or the gamma pass rate of the current radiotherapy plan falling below a set threshold. In response to determining that the radiotherapy device has the preset type of failure, the plan verification module 230 may terminate the verification of the target radiotherapy plan and verify a next target radiotherapy plan in the radiotherapy plan temporary list, which improves the efficiency of the batch verification of the target radiotherapy plans, and prevents the minor failure from affecting an entire process of the batch verification, thereby saving unnecessary time for processing the minor failure, ensuring the smoothness of the entire process of automatic verification, and improving the execution efficiency.

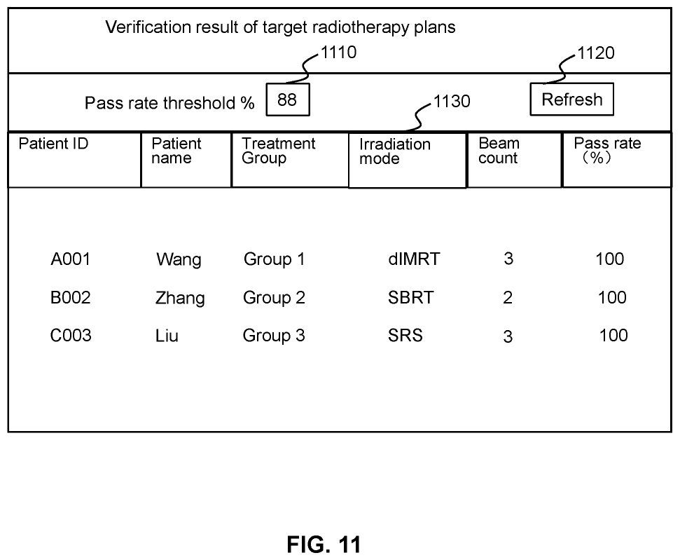

In some embodiments, the plan verification module 230 may generate a verification result for review by a user (e.g., the radiotherapy operator, a patient's attending doctor, etc.) after each target radiotherapy plan is verified, or after all target radiotherapy plans in the radiotherapy plan temporary list are verified. Merely by way of example, the verification result may include a verification result of each target radiotherapy plan. For example, as shown in FIG. 11, the verification result of each target radiotherapy plan may include a patient name, a patient ID (identification), a treatment group (i.e., a field group), a beam count, a treatment type, a pass rate (i.e., the gamma pass rate), etc. In some embodiments, the plan verification module 230 may send the verification result to a user terminal (e.g., the terminal 140) for review by the user. In some embodiments, the plan verification module 230 may save the verification result in a storage device (e.g., the storage device 130) for review by the user at any time.

According to the embodiments of the present disclosure, the fault analysis result is generated by automatically performing the fault analysis on the target radiotherapy plan that does not meet the first preset condition, so that a radiotherapy operator can handle the fault of the radiotherapy device based on the fault analysis result, which reduces the difficulty of fault determination, and saves time and energy of the radiotherapy operator. Accordingly, the radiotherapy operator can quickly resolve the fault of the radiotherapy device, thereby ensuring the normal progress of the verification of the target radiotherapy plans.

FIG. 7 is a schematic diagram illustrating an exemplary process for verifying target radiotherapy plans in a radiotherapy plan temporary list according to some embodiments of the present disclosure.

In some embodiments, in the radiotherapy plan temporary list, the target radiotherapy plans may be divided into radiotherapy plan groups based on at least one feature parameter of each of the target radiotherapy plans. For example, as shown in FIG. 7, the target radiotherapy plans may be divided into a group 1 including target radiotherapy plans of dynamic IMRT group 1 and a group 2 including target radiotherapy plans of static IMRT based on the radiotherapy type. In the radiotherapy plan temporary list, target radiotherapy plans of a same group may be arranged together. For example, as shown in FIG. 7, target radiotherapy plans 9, 10, 11, 12, 13, and 14 of the group 1 may be arranged together; and target radiotherapy plans 15, 16, 17, 18, and 19 of the group 2 may be arranged together. In some embodiments, the target radiotherapy plans of the same group may be arranged randomly or arranged in a specific order. For example, the target radiotherapy plans of the same group may be arranged based on a field size (e.g., arranged in a descending order) and/or a field location. Merely by way of example, the field location of each target radiotherapy plan is represented by a distance between the irradiation region and the target region (e.g., a tumor), and the target radiotherapy plans in the same group are arranged based on the corresponding distances in an ascending order. The irradiation region refers to a region of a treatment subject irradiated by radiation. The target region refers to a region of the treatment subject where a lesion is located.

Further, for each of the radiotherapy plan groups, the plan verification module 230 may sequentially verify one or more target radiotherapy plans in the radiotherapy plan group. For example, as shown in FIG. 7, the plan verification module 230 may sequentially verify the target radiotherapy plans 9, 10, 11, 12, 13, and 14 of the group 1. Whenever one of the one or more target radiotherapy plans in the radiotherapy plan group passes the verification, the plan verification module 230 may determine whether the radiotherapy plan group meets a second preset condition. For example, the second preset condition may be whether a count or proportion of target radiotherapy plans in the radiotherapy plan group that pass the verification exceeds a threshold. If the count or the proportion of target radiotherapy plans in the radiotherapy plan group that pass the verification does not exceed the threshold, the plan verification module 230 may determine that the radiotherapy plan group does not meet the second preset condition. In response to determining that the radiotherapy plan group does not meet the second preset condition, the plan verification module 230 may verify a next target radiotherapy plan in the radiotherapy plan group. If the count or the proportion of target radiotherapy plans in the radiotherapy plan group that pass the verification exceeds the threshold, the plan verification module 230 may determine that the radiotherapy plan group meets the second preset condition. In response to determining that the radiotherapy plan group meets the second preset condition, the plan verification module 230 may determine that other target radiotherapy plans in the radiotherapy plan group pass the verification.

For example, as shown in FIG. 7, when the target radiotherapy plan 9 of the group 1 passes the verification, the plan verification module 230 may determine that the proportion of target radiotherapy plans of the group 1 that pass the verification does not exceed the threshold (e.g., 20%), and verify the target radiotherapy plan 10. When the target radiotherapy plan 10 passes the verification, the plan verification module 230 may determine that the proportion of the target radiotherapy plans of the group 1 that pass the verification exceeds the threshold (e.g., 20%), and determine that the target radiotherapy plans 11, 12, 13, and 14 pass the verification.

Since target radiotherapy plans in the same group have the same or similar characteristics, the verification result of other target radiotherapy plans can be estimated when some target radiotherapy plans pass the verification. In this way, some target radiotherapy plans can be verified without actually executing the target radiotherapy plans, and the verification efficiency can be improved.

In some embodiments, the target radiotherapy plans of the same group may be arranged based on field size (e.g., arranged in the descending order) and/or the distance between the irradiation region and the target region (e.g., arranged from near to far). If top target radiotherapy plans (i.e., target radiotherapy plans corresponding to a large field size and/or a small distance between the irradiation region and the target region in a radiotherapy plan group pass the verification, other target radiotherapy plans (i.e., target radiotherapy plans corresponding to a small field size and/or a large distance between the irradiation region and the target region) in the radiotherapy plan group are supposed to pass the verification. Therefore, it can be directly determined that the other target radiotherapy plans in the radiotherapy plan group pass the verification, thereby improving the verification efficiency.

It should be understood that the grouping manner shown in FIG. 7 is merely provided for illustration purposes, and not intended to be limiting. Merely by way of example, the target radiotherapy plans may be grouped into multiple groups corresponding to different field size ranges. Optionally, the target radiotherapy plans in each group may be arranged based on their respective field sizes in a descending order.

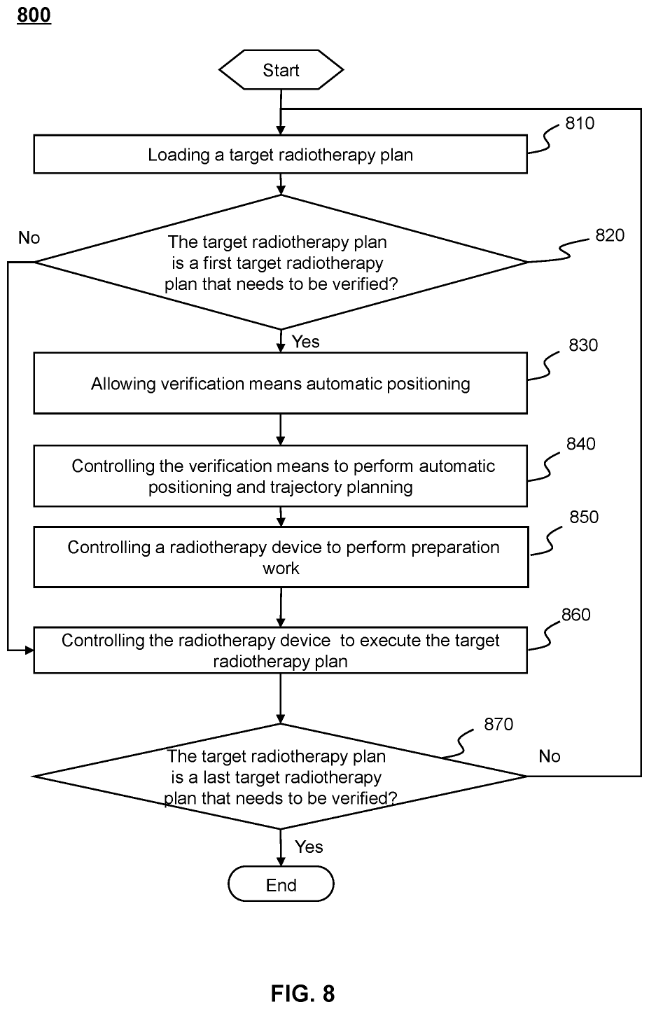

FIG. 8 is a flowchart illustrating an exemplary process 800 of radiotherapy plan verification according to some embodiments of the present disclosure. As shown in FIG. 8, in some embodiments, the process 800 may include the following operations. In some embodiments, the process 800 may be implemented by the processing device 120 or the plan verification module 230. In some embodiments, operations shown in the process 800 may be performed by the processing device 120 to implement operation 330 to verify the target radiotherapy plans in the radiotherapy plan temporary list.

In 810, a target radiotherapy plan may be loaded.

At the beginning, the processing device 120 may load the target radiotherapy plan that currently needs to be verified from the radiotherapy plan temporary list.

In 820, whether the target radiotherapy plan is a first target radiotherapy plan that needs to be verified may be determined.

If the target radiotherapy plan is the first target radiotherapy plan that needs to be verified, the processing device 120 may perform operation 830. If the target radiotherapy plan is not the first target radiotherapy plan that needs to be verified, the processing device 120 may perform operation 860.

In 830, verification means automatic positioning may be allowed.

The processing device 120 may set the radiotherapy device (e.g., the radiotherapy device 110) to allow the verification means automatic positioning, and then perform operation 840.

In 840, a verification means may be controlled to perform automatic positioning and trajectory planning.

The processing device 120 may control the verification means of the radiotherapy device to perform the automatic positioning and the trajectory planning. The trajectory planning refers to planning irradiation trajectories of radiation beams.

In 850, the radiotherapy device may be controlled to perform preparation work.

After the automatic positioning is completed, the processing device 120 may control the radiotherapy device to perform preparation work, e.g., parameter adjustment, etc.

In 860, the radiotherapy device may be controlled to execute the target radiotherapy plan.

After the preparation work of the radiotherapy device is completed, the processing device 120 may control the radiotherapy device to execute the target radiotherapy plan.

In 870, whether the target radiotherapy plan is a last target radiotherapy plan that needs to be verified may be determined.

After the target radiotherapy plan is executed, the processing device 120 may determine whether the target radiotherapy plan is the last target radiotherapy plan in the radiotherapy plan temporary list. If the target radiotherapy plan is the last target radiotherapy plan in the radiotherapy plan temporary list, the processing device 120 may end the radiotherapy plan verification process. If the target radiotherapy plan is not the last target radiotherapy plan in the radiotherapy plan temporary list, the processing device 120 may load a next target radiotherapy plan in the radiotherapy plan temporary list, i.e., to perform operation 810 again.

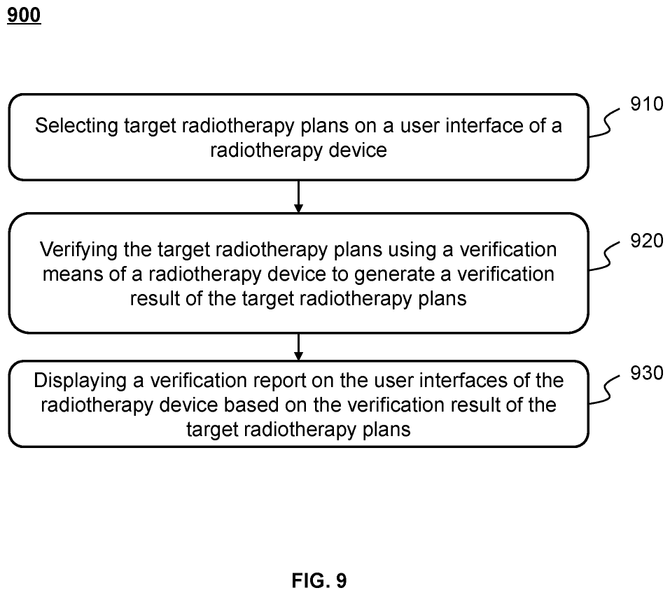

FIG. 9 is a flowchart illustrating an exemplary process 900 of radiotherapy plan verification according to some embodiments of the present disclosure. As shown in FIG. 9, the process 900 may include the following operations. In some embodiments, the process 900 may be implemented by the processing device 120 or one or more modules illustrated in FIG. 2.

In 910, target radiotherapy plans may be selected on a user interface of a radiotherapy device (e.g., the radiotherapy device 110). These target radiotherapy plans may be radiotherapy plans that need to be verified.

In some embodiments, the processing device 120 may select the radiotherapy plans from a radiotherapy plan list displayed in the user interface based on a user instruction of a user as the target radiotherapy plans that need to be verified. More descriptions of selecting the target radiotherapy plans may be found elsewhere in the present disclosure (e.g., operation 310 and the descriptions thereof).

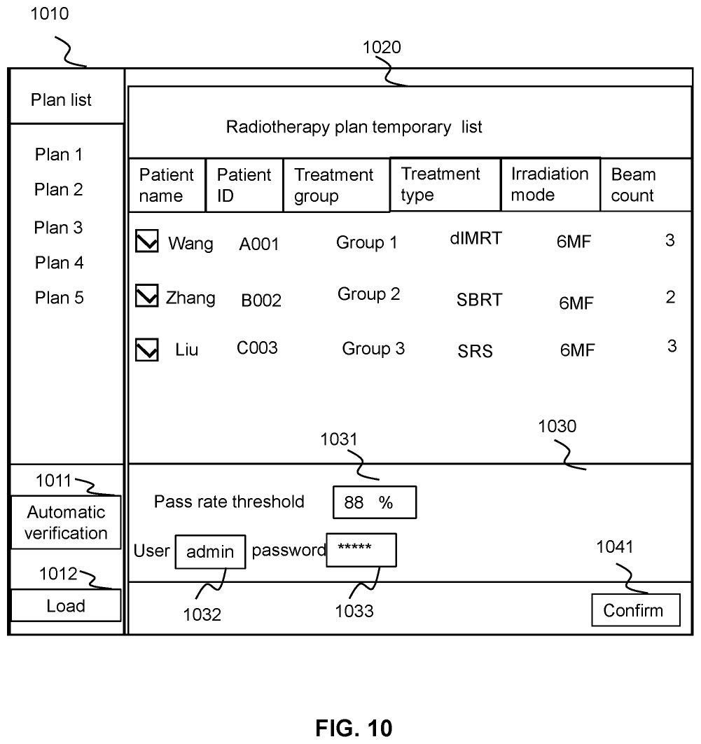

Merely by way of example, FIG. 10 is a schematic diagram illustrating an exemplary user interface for selecting target radiotherapy plans according to some embodiments of the present disclosure. As shown in FIG. 10, the user may select radiotherapy plans in a radiotherapy plan list 1010. The selected radiotherapy plans may be displayed in a radiotherapy plan temporary list 1020 as the target radiotherapy plans. In some embodiments, the user may select to perform automatic verification by clicking a button 1011, so that all radiotherapy plans in the radiotherapy plan list 1010 may be added to the radiotherapy plan temporary list 1020 as the target radiotherapy plans. In some embodiments, after the user selects the target radiotherapy plans, the user may click a button 1012 to load these selected target radiotherapy plans to the radiotherapy plan temporary list 1020.

In 920, the target radiotherapy plans may be verified using a verification means of a radiotherapy device to generate a verification result of the target radiotherapy plans.

In some embodiments, the processing device 120 may generate a radiotherapy plan temporary list based on the target radiotherapy plans, and then verify the target radiotherapy plans in batches based on the radiotherapy plan temporary list using a verification means of the radiotherapy device. More descriptions of generating the radiotherapy plan temporary list and performing the batch verification may be found elsewhere in the present disclosure (e.g., operations 320 and 330 and the descriptions thereof).

Merely by way of example, as shown in FIG. 10, each target radiotherapy plan in the radiotherapy plan temporary list 1020 may include the following information: a patient name, a patient ID, a treatment group, a treatment type, an irradiation mode, and a beam count. The user may enter, in a region 1030, additional information required for the execution of each target radiotherapy plan. For example, the user may enter a pass rate threshold in an input box 1031. As shown in FIG. 10, the pass rate threshold may be 88%. As another example, the user may enter a user name and a password in input boxes 1032 and 1033, respectively, to verify a user identity. After all the information required for the execution of all the target radiotherapy plans is entered, the user may confirm the start of the verification of the target radiotherapy plans by clicking a button 1041.

In some embodiments, after the target radiotherapy plans are verified, the processing device 120 may generate the verification result of the target radiotherapy plans and display the verification result to the user through various means.

Merely by way of example, FIG. 11 is a schematic diagram illustrating an exemplary user interface displaying a verification result of target radiotherapy plans according to some embodiments of the present disclosure. As shown in FIG. 11, the content in a text box 1110 may be a preset pass rate threshold, which may correspond to the input value in the input box 1031 in FIG. 10. A button 1120 may be a refresh button, and the user may refresh the display of the user interface by clicking the button 1120. A list 1130 may be the verification result of target radiotherapy plans, and each item in the verification result may include the following information: a patient ID, a patient name, a treatment group, an irradiation, a beam count, a pass rate (i.e., a gamma pass rate).

In 930, a verification report may be displayed on the user interface of the radiotherapy device based on the verification results of the target radiotherapy plans. In some embodiments, the processing device 120 may generate the verification report based on the verification result of the target radiotherapy plans, and display the verification report on the user interface of the radiotherapy device. The verification report may include various information related to the execution of the target radiotherapy plans, e.g., execution environmental information, plan basic information, plan analysis information, an analysis standard, a dose map, etc.

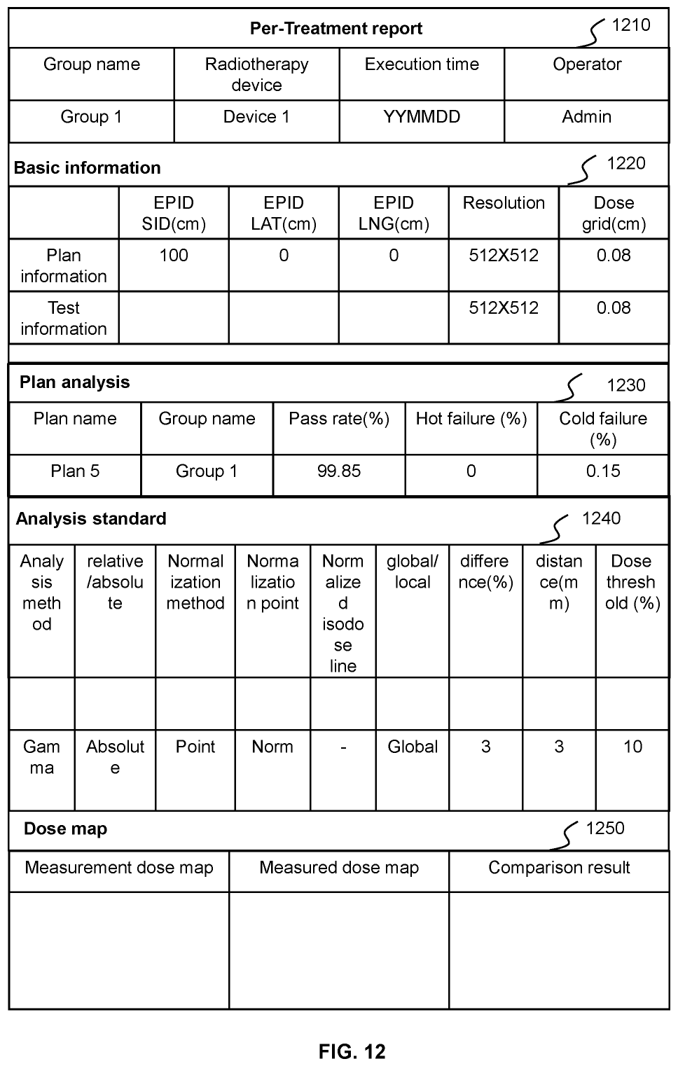

Merely by way of example, FIG. 12 is a schematic diagram illustrating an exemplary verification report of a target radiotherapy plan according to some embodiments of the present disclosure. As shown in FIG. 12, the verification report may be referred to as a pre-treatment report. A table 1210 in the pre-treatment report displays the execution environmental information of the plan verification of the target radiotherapy plan 5, and the execution environmental information includes a group name, a radiotherapy device, execution time, and an operator. A table 1220 in the pre-treatment report displays the basic information of the target radiotherapy plan 5, and the basic information includes plan information and test information each of which includes the following information: EPID SID (Security Identifier), EPID LAT (latitude), EPID LNG (longitude), resolution, and a dose grid. A table 1230 in the pre-treatment report displays the plan analysis information of the target radiotherapy plan 5, and the plan analysis information includes a plan name, a group name, a pass rate, a hot failure, and a cold failure. A table 1240 in the pre-treatment report displays the analysis standard of the target radiotherapy plan 5, and the analysis standard includes an analysis method, relative/absolute, a normalization method, a normalization point, a normalized isodose line, global/local, a difference, a distance, and a dose threshold. A table 1250 in the pre-treatment report displays the dose map of the target radiotherapy plan 5, and the dose map includes a measurement dose (i.e., a theoretically received dose) map, a measured dose (i.e., an actually received dose) map, and a comparison result of the measurement dose map and the measured dose map.

It should be noted that the above descriptions of the processes 300, 600, 800, and 900 are only for examples and illustrations, and do not limit the scope of application of the present disclosure. For those skilled in the art, various modifications and changes can be made to the processes 300, 600, 800, and 900 under the guidance of the present disclosure. However, such modifications and changes remain within the scope of the present disclosure. For example, in operation 810, all target radiotherapy plans in the radiotherapy plan temporary list may be loaded at one time. In some embodiments, a process may be accomplished with one or more additional operations not described, and/or without one or more of the operations discussed. Additionally, the order in which the operations of a process described above is not intended to be limiting.

Having thus described the basic concepts, it may be rather apparent to those skilled in the art after reading this detailed disclosure that the forward detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the examples of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specifications are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the forward description of embodiments of the present disclosure, various features are sometimes grouped together in a single embodiment, figure, or description thereof for the purpose of streamlining the disclosure aiding in the understanding of one or more of the various inventive embodiments. This method of disclosure embodiment, however, is not to be interpreted as reflecting an intention that the claimed object matter requires more features than are expressly recited in each claim. Rather, inventive lie in less than all features of a single forwarding disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for radiotherapy plan verification, implemented on a computing device including at least one processor, the method comprising:
obtaining target radiotherapy plans which need to be verified;
generating a radiotherapy plan temporary list based on the target radiotherapy plans; and
verifying the target radiotherapy plans in batches based on the radiotherapy plan temporary list using at least one verification means of at least one radiotherapy device.

2. The method of claim 1, wherein the obtaining target radiotherapy plans which need to be verified includes:
determining the target radiotherapy plans from a plurality of radiotherapy plans.

3. The method of claim 2, wherein the determining the target radiotherapy plans from the plurality of radiotherapy plans includes:
presenting, via a user terminal, the plurality of radiotherapy plans; and
receiving, via the user terminal, a user instruction for selecting the target radiotherapy plans from the plurality of radiotherapy plans.

4. The method of claim 3, wherein
the user terminal is further directed to present a filtering element for filtering the plurality of radiotherapy plans using one or more filtering conditions,
the user instruction is input via the filtering element presented by the user terminal, and
the one or more filtering conditions include at least one of a treatment part, a treatment subject, or a treatment type.

5. The method of claim 2, wherein the determining the target radiotherapy plans from the plurality of radiotherapy plans includes:
obtaining status information of the at least one radiotherapy device;
for each of the plurality of radiotherapy plans,
determining planned radiotherapy parameters of the radiotherapy plan; and
determining a risk coefficient of the radiotherapy plan based on the planned radiotherapy parameters and the status information of a corresponding radiotherapy device; and
selecting the target radiotherapy plans from the plurality of radiotherapy plans based on the risk coefficients of the plurality of radiotherapy plans.

6. The method of claim 1, wherein the generating the radiotherapy plan temporary list based on the target radiotherapy plans includes:
for each of the target radiotherapy plans, determining a starting irradiation angle and an ending irradiation angle of radiation beams of the target radiotherapy plan;
determining a first sorting result of the target radiotherapy plans based on the starting irradiation angle and the ending irradiation angle of each of the target radiotherapy plans; and
generating the radiotherapy plan temporary list in which the target radiotherapy plans are sorted according to the first sorting result.

7. The method of claim 1, wherein the generating the radiotherapy plan temporary list based on the target radiotherapy plans includes:
for each of the target radiotherapy plans,
determining irradiation angles of radiation beams of the target radiotherapy plan;
determining a second sorting result of the radiation beams of the target radiotherapy plan based on the irradiation angles of the radiation beams of the target radiotherapy plan; and
generating the radiotherapy plan temporary list in which the radiation beams of the target radiotherapy plan are sorted according to the second sorting result.

8. The method of claim 1, wherein the verifying the target radiotherapy plans in batches based on the radiotherapy plan temporary list using the at least one verification means of the at least one radiotherapy device includes:
controlling the at least one verification means to perform automatic positioning; and
sequentially verifying the target radiotherapy plans in the radiotherapy plan temporary list, each of the target radiotherapy plans being verified using the verification means of one of the at least one radiotherapy device after automatic positioning.

9. The method of claim 8, wherein the sequentially verifying the target radiotherapy plans in the radiotherapy plan temporary list includes:
for each of the target radiotherapy plans in the radiotherapy plan temporary list,
controlling the verification means that has experienced the automatic positioning to collect test data during an execution process of the target radiotherapy plan;
determining whether the target radiotherapy plan meets a first preset condition based on the test data; and
in response to determining that the target radiotherapy plan meets the first preset condition, verifying a next target radiotherapy plan in the radiotherapy plan temporary list; or
in response to determining that the target radiotherapy plan does not meet the first preset condition, performing fault analysis on a corresponding radiotherapy device to obtain a fault analysis result.

10. The method of claim 9, wherein the performing fault analysis on the corresponding radiotherapy device to obtain the fault analysis result includes:
obtaining at least one device parameter of the radiotherapy device; and
generating the fault analysis result of the radiotherapy device based on the at least one device parameter.

11. The method of claim 10, wherein the generating the fault analysis result of the radiotherapy device based on the at least one device parameter includes:
generating the fault analysis result of the radiotherapy device by using a fault analysis model to process the at least one device parameter, the fault analysis model being a trained machine learning model.

12. The method of claim 9, wherein in response to determining that the target radiotherapy plan does not meet the first preset condition, the method further includes:
determining whether the radiotherapy device has a preset type of fault based on the fault analysis result; and
in response to determining that the radiotherapy device has the preset type of fault, terminating or skipping the verification of the target radiotherapy plan and verifying a next target radiotherapy plan in the radiotherapy plan temporary list.

13. The method of claim 1, wherein
in the radiotherapy plan temporary list, the target radiotherapy plans are divided into radiotherapy plan groups based on at least one feature parameter of each of the target radiotherapy plans, and
the verifying the target radiotherapy plans in batches includes:
for each of the radiotherapy plan groups,
sequentially verifying one or more target radiotherapy plans in the radiotherapy plan group;
whenever one of the one or more target radiotherapy plans in the radiotherapy plan group passes the verification, determining whether the radiotherapy plan group meets a second preset condition; and
in response to determining that the radiotherapy plan group meets the second preset condition, determining that other target radiotherapy plans in the radiotherapy plan group pass the verification.

14. The method of claim 13, wherein the at least one feature parameter includes at least one of a radiotherapy type, a field location, or a field size.

15. The method of claim 1, wherein
the at least one radiotherapy device includes multiple radiotherapy devices, and
the verifying the target radiotherapy plans in batches based on the radiotherapy plan temporary list through the at least one verification means of the at least one radiotherapy device includes:
determining reference information of each of the radiotherapy devices, the reference information including at least one of reservation information or historical usage information;
assigning the target radiotherapy plans in the radiotherapy plan temporary list to the radiotherapy devices for verification based on the reference information of each of the radiotherapy devices.

16. The method of claim 1, wherein the at least one verification means of the at least one radiotherapy device includes at least one of an electronic portal imaging device (EPID) or a radiation detector.

17. A system, comprising:
at least one storage device including a set of instructions; and
at least one processor in communication with the at least one storage device, wherein when executing the set of instructions, the at least one processor causes the system to perform operations including:
obtaining target radiotherapy plans which need to be verified;
generating a radiotherapy plan temporary list based on the target radiotherapy plans; and
verifying the target radiotherapy plans in batches based on the radiotherapy plan temporary list using at least one verification means of at least one radiotherapy device.

18. The method of claim 17, wherein the obtaining target radiotherapy plans which need to be verified includes:
determining the target radiotherapy plans from a plurality of radiotherapy plans.

19. The system of claim 18, wherein the determining the target radiotherapy plans from the plurality of radiotherapy plans includes:
presenting, via a user terminal, the plurality of radiotherapy plans; and
receiving, via the user terminal, a user instruction for selecting the target radiotherapy plans from the plurality of radiotherapy plans.

20. A non-transitory computer readable medium, comprising executable instructions that, when executed by at least one processor, direct the at least one processor to perform a method, the method comprising:
obtaining target radiotherapy plans which need to be verified;
generating a radiotherapy plan temporary list based on the target radiotherapy plans; and
verifying the target radiotherapy plans in batches based on the radiotherapy plan temporary list using at least one verification means of at least one radiotherapy device.
